(19) United States
(12) Patent Application Publication
(10) Pub. No.: US 2022/0257535 A1
(43) Pub. Date: Aug. 18, 2022

(54) INK FORMULATION AND MANUFACTURE THEREOF

(71) Applicant: David Bradley Arnold, Albuquerque, NM (US)

(72) Inventor: David Bradley Arnold, Albuquerque, NM (US)

(73) Assignee: Apothecary Ink LLC, Albuquerque, NM (US)

(21) Appl. No.: 17/399,000

(22) Filed: Aug. 10, 2021

Related U.S. Application Data
(60) Provisional application No. 63/150,049, filed on Feb. 16, 2021.

(51) Int. Cl. A61K 31/135; A61K 31/045; A61K 8/19; A61K 8/34; A61Q 1/02

(52) U.S. Cl. CPC A61K 31/135; A61K 31/045; A61Q 1/025; A61K 8/345; A61K 8/19

(57) ABSTRACT

An ink for marking living tissue, the ink comprising: a carrier; an analgesic; an antiseptic; an antihistamine; and a coloring agent. A method of preparing an ink formulation for marking living tissue, comprising the steps of: mixing the pharmaceutically accepted carrier, analgesic, antiseptic, and antihistamine to form a carrier preservative compound; adding the pharmaceutically accepted coloring agent to the carrier preservative compound; mixing the pharmaceutically accepted coloring agent to the carrier preservative compound to form said ink formulation. A carrier preservative compound for removing ink from living tissue comprising: a carrier; an analgesic; an antiseptic; and an antihistamine.

### CROSS-REFERENCE TO RELATED APPLICATION

This application claims the benefit of U.S. Provisional Patent Application Ser. No. 63/150,049 filed Feb. 16, 2021, titled “INK FORMULATION AND MANUFACTURE THEREOF” and the subject matter thereof is incorporated herein by reference thereto.

### TECHNICAL FIELD

The present invention relates to an ink formulation, and more specifically, an ink formulation that can be used for marking living tissue, along with a method for preparing the ink.

### BACKGROUND ART

Tattooing has been around for thousands of years. The inks and dyes that were historically used were derived from natural substances and comprised a heterogeneous suspension of pigmented particles and other impurities. Tattoos are made by applying tattoo ink on living tissue, the dermis, where the ink remains permanently. The tattoo process involves inserting the ink through the skin by an alternating pressure-suction action caused by the elasticity of the skin in combination with the up-and-down movement of tattoo needles. A carrier in the tattoo in caries the pigment which has been introduced into the skin and allows the ink to diffuse throughout the tissue. The pigment is insoluble and remains largely in the dermis.

The tattoo process can be very painful as it includes a large number of small needle pricks to the skin. The tattoo process also results in inflammation and irritation. There are a large number of tattoo inks available. Additionally, many tattoo inks are created by the tattoo artists themselves, which can result in varying qualities of ink. However, the primary difference in one tattoo ink to the next is the color. There are currently not any tattoo inks that provide any sort of pain relief while also decreasing inflammation and irritation.

U.S. Pat. No. 7,699,917 B1 entitled, “Selectively Alterable Intermittent Tattoo Ink and System,” to Pagnotta, U.S. Pat. No. 8,709,142 B2 entitled, “Ink Formulation and Manufacture Thereof,” to Story et al., U.S. Pat. No. 9,034,087 B2 entitled, “Ink Formulation and Manufacture Thereof,” to Story et al., U.S. Pat. No. 7,510,603 B2 entitled, “Tattooing Ink,” to Michel, U.S. Pat. No. 6,013,122 entitled, “Tattoo Inks,” to Klitzman et al. each disclose tattoo inks. However, there are shortcomings in the prior art tattoo inks in that some are difficult to manufacturer and do not contain an analgesic, antihistamine, and/or antiseptic.

The present invention overcomes the shortcomings contained in the prior art by providing a tattoo ink that provides pain relief to the skin while also decreasing inflammation and irritation at the tattoo site. The present invention is also cheap, easy to use, and easy to produce.

Certain embodiments of the invention have other steps or elements in addition to or in place of those mentioned above. The steps or element will become apparent to those skilled in the art from a reading of the following detailed description.

### DETAILED DESCRIPTION OF THE INVENTION

The following embodiments are described in sufficient detail to enable those skilled in the art to make and use the invention. It is to be understood that other embodiments would be evident based on the present disclosure, and that system, process, or mechanical changes may be made without departing from the scope of the present invention.

In the following description, numerous specific details are given to provide a thorough understanding of the invention. However, it will be apparent that the invention may be practiced without these specific details. To avoid obscuring the present invention, some well-known system configurations, and process steps are not disclosed in detail. Alternate embodiments have been included throughout, and the order of such are not intended to have any other significance or provide limitations for the present invention.

The present invention provides ink for marking living tissue, the ink comprising: a carrier; an analgesic; an antiseptic; an antihistamine; and a coloring agent. The present invention further comprises a method of preparing an ink formulation for marking living tissue, comprising the steps of: mixing the pharmaceutically accepted carrier, analgesic, antiseptic, and antihistamine to form a carrier preservative compound; adding the pharmaceutically accepted coloring agent to the carrier preservative compound; mixing the pharmaceutically accepted coloring agent to the carrier preservative compound to form said ink formulation. The present invention further comprises a carrier preservative compound for removing ink from living tissue comprising: a carrier; an analgesic; an antiseptic; and an antihistamine.

An ink for marking living tissue, referred to as a tattoo ink, with the following constituents is particularly advantageous: the carrier is propylene glycol; the analgesic is benzyl alcohol; the antiseptic is ethanol; the antihistamine is diphenhydramine; and the coloring agent is a pharmaceutically accepted pigment.

A tattoo ink, whose constituents are present in the following compositions is particularly advantageous: the propylene glycol is represented in a proportion of 40% to 60% v/v; the benzyl alcohol is represented in proportion of 9% to 35% v/v; the ethanol is represented in proportion of 8% to 25% v/v; and the diphenhydramine is represented in a proportion of 10 mg/mL to 40 mg/mL of the total formulation by volume. The diphenhydramine is expressed in mg/mL because it is a powder, while the other chemicals of the ink are liquid. The user can easily measure out an amount by mass of the diphenhydramine in order to reach the desired concentration of same.

Preferably, the tattoo ink of the present invention is created by the following steps. First, the pharmaceutically accepted carrier, analgesic, antiseptic, and antihistamine are added to a 10 ML food safe, BPA free, non-leeching bottle with a stainless steel food grade ball bearing located in the bottle. The bottle is shaken after the first set of chemicals are added to form a carrier preservative compound. A pharmaceutically accepted coloring agent is then added to the carrier preservative compound. The coloring agent is added in small amounts, and then the mixture is mixed by shaking the bottle. The consistency and color of the tattoo ink can be changed as desired by adding additional coloring agent and shaking the bottle. Coloring agents, or pigments, vary in compositions, weights by volume, and specific gravities. As such, there are no set amounts for all pigments and that is left to the user. Additionally, some pigments may require for additional amounts of the carrier to be added in order to affect the viscosity of the tattoo ink.

With respect chemicals listed above that comprise an advantageous tattoo ink of the present invention, propylene glycol is the carrier. Benzyl alcohol is the analgesic as it is classified as a pediculicide (anti-parasite), and it decreases bleeding, inflammation, and swelling at the site, and acts as an analgesic. Ethanol is the antiseptic. Diphenhydramine (trade name Benadryl) is classified as an antihistamine and acts to decrease inflammation, irritation, and pain at the tattoo site.

The combination of a carrier, an analgesic, an antihistamine, and an antihistamine provides maximum benefit to a person receiving the tattoo by providing pain relief during the tattoo process and reducing swelling/irritation at the tattoo site.

In alternate embodiments of the present invention, the tattoo ink comprises any number of the chemicals comprising a carrier, an analgesic, an antiseptic, an antihistamine, and a coloring agent, in any combination, ratios, and percentages.

In an alternate embodiment of the present invention, the carrier is any pharmaceutically accepted carrier that can allow the tattoo ink to be tattooed onto living tissue.

In an alternate embodiment of the present invention, the analgesic is any pharmaceutically accepted substance that provides numbing and/or pain relief.

In an alternate embodiment of the present invention, the antiseptic is any pharmaceutically accepted substance that prevents the growth of disease-causing microorganisms.

In an alternate embodiment of the present invention, the antihistamine is any pharmaceutically accepted substance that decreases inflammation and/or irritation.

In an alternate embodiment of the present invention, the coloring agent is any pharmaceutically accepted pigment.

In an alternate embodiment of the present invention, the ink of the present invention can be used for recreational and cosmetic ink and can also be used in microblading. Microblading is a series of small blades used to rake over areas such as the eyebrows to create microscopic cuts, and deposit inks.

In an alternate embodiment of the invention, witch hazel is added to the tattoo ink formulation. The witch hazel decreases soreness, redness, and swelling.

The carrier preservative compound of the present invention may also be used for tattoo removal. Tattoos work by injecting ink under the skin. The ink is then “encased” by white blood cells, which try to remove the ink from the body, but cannot due to the size of the ink. Tattoo removal methods include laser and saline (saltwater) treatments. Tattoo removal is typically painful, expensive, has varying degrees of success, and can take many separate treatments.

Laser treatment works by breaking apart the ink, thus allowing the body to absorb and ultimately excrete the smaller ink particles. Saline treatments are applied over an existing tattoo, usually with a tattoo gun, drawing the ink to the surface. Multiple saline treatments are necessary, and the area will scab over after each treatment. Saline treatments are not as effective as laser treatments; however, saline treatment is becoming more common when removing ink from eyebrows (microblading) and seems to be growing in popularity for that market segment.

The carrier preservative compound of the present invention can be used for tattoo removal by using saline as a carrier and not adding the pigment. The carrier preservative compound of the present invention provides additional pain relief and reduction of swelling while removing the ink from the body. The carrier preservative compound also provides better results in the removal of the ink, resulting in fewer treatments required.

The carrier preservative compound with the following constituents is particularly advantageous for the removal of tattoo ink: the carrier is saline; the analgesic is benzyl alcohol; the antiseptic is ethanol; and the antihistamine is diphenhydramine.

The previous embodiments are described in sufficient detail to enable those skilled in the art to make and use the invention. It is to be understood that other embodiments would be evident based on the present disclosure, and that system, process, or mechanical changes may be made without departing from the scope of the present invention.

In the previous description, numerous specific details and examples are given to provide a thorough understanding of the invention. However, it will be apparent that the invention may be practiced without these specific details and specific examples. While the invention has been described in conjunction with a specific best mode, it is to be understood that many alternatives, modifications, and variations will be apparent to those skilled in the art in light of the foregoing description. Accordingly, it is intended to embrace all such alternatives, modifications, and variations that fall within the scope of the included claims. All matters previously set forth herein are to be interpreted in an illustrative and non-limiting sense.

What is claimed is:

1. An ink for marking living tissue, the ink comprising:
a carrier;
an analgesic;
an antiseptic;
an antihistamine; and
a coloring agent.

2. The ink formulation of claim 1, wherein said carrier is propylene glycol.

3. The ink formulation of claim 2, wherein said propylene glycol comprises 40% to 60% v/v of the total formulation by volume.

4. The ink formulation of claim 1, wherein said analgesic is benzyl alcohol.

5. The ink formulation of claim 4, wherein said benzyl alcohol comprises 9% to 35% v/v of the total formulation by volume.

6. The ink formulation of claim 1, wherein said antiseptic is ethanol.

7. The ink formulation of claim 6, wherein said ethanol comprises 8% to 25% v/v of the total formulation by volume.

8. The ink formulation of claim 1, wherein said antihistamine is diphenhydramine.

9. The ink formulation of claim 8, wherein said diphenhydramine comprises 10 mg/mL to 40 mg/mL of the total formulation by volume.

10. The ink formulation of claim 1, wherein said coloring agent is a pharmaceutically accepted pigment.

11. A method of preparing an ink formulation for marking living tissue, comprising the steps of:
mixing the pharmaceutically accepted carrier, analgesic, antiseptic, and antihistamine to form a carrier preservative compound;
adding the pharmaceutically accepted coloring agent to the carrier preservative compound; and
mixing the pharmaceutically accepted pigment to the carrier preservative compound to form said ink formulation.

12. The method of preparing an ink formulation of claim 11, wherein incremental amounts of the pharmaceutically accepted coloring agent is added to the carrier preservative compound and mixed.

13. A carrier preservative compound for removing ink from living tissue comprising:
a carrier;
an analgesic;
an antiseptic; and
an antihistamine.

14. The carrier preservative compound formulation of claim 13, wherein said carrier is saline.

15. The carrier preservative compound of claim 13, wherein said analgesic is benzyl alcohol.

16. The carrier preservative compound of claim 13, wherein said antiseptic is ethanol.

17. The carrier preservative compound of claim 13, wherein said antihistamine is diphenhydramine.
